(19) United States
(12) Patent Application Publication
(10) Pub. No.: US 2026/0145839 A1
(43) Pub. Date: May 28, 2026

(54) METHOD AND APPARATUS FOR AN AIR-EXPELLING, BUBBLE-FREE CAPSULE

(71) Applicant: Bing Baksh, Placentia, CA (US)

(72) Inventor: Bing Baksh, Placentia, CA (US)

(73) Assignee: Vita-Herb Nutriceuticals, Inc., Placentia, CA (US)

(21) Appl. No.: 18/958,367

(22) Filed: Nov. 25, 2024

(51) Int. Cl. B65D 8/00; B65B 3/04; B65B 3/18; B65D 51/16

(52) U.S. Cl. CPC B65D 11/02; B65B 3/04; B65B 3/18; B65D 51/16

(57) ABSTRACT

A method and apparatus for a two-piece capsule configured to expel air and reduce bubbles within a composition. The invention features a capsule with a cap and body, where the cap includes an insert with a dome shape designed to compress against the meniscus of a fluid composition or powder. This compression minimizes trapped air and enhances the capsule's structural integrity and longevity. Additionally, the capsule may employ a sealing element to prevent leakage, ensuring a stable, bubble-free environment for the contents. The capsule's design allows for efficient filling and reduces oxygen exposure, thus preserving the quality of the encapsulated material. The invention can be used for both powder and liquid encapsulations, enhancing shelf life and stability by significantly reducing internal air and maximizing fill volume.

### BACKGROUND

The invention relates generally to the field of two-piece capsules and is more particularly, but not by way of limitation, directed to methods and apparatus for providing a leak-proof seal for powder and liquid encapsulation systems. Capsules are manufactured to encapsulate liquids or powders for consumer consumption. The basic shape and challenges associated with such capsules have remained the same for quite some time. Capsules of this sort have been elongated in shape and have rounded ends to facilitate easy consumption. A problem with this approach is that when filling the capsules air is introduced into the capsule and that degrades the quality of the liquid or powder contained within the capsule which shortens the shelf life and/or effectiveness of the liquid or powder contained within the capsule. Thus, there is a need for a method and apparatus, an air-expelling and bubble-free capsule. FIG. 1 illustrates the components of a prior art capsule. The capsule comprises a body (100) and a cap (104). The cap (104) typically has a wider diameter than the body (100) which enables the body (100), which is elongated, to fit inside the cap (104) thereby creating an overlap (102) between the body (100) and the cap (104). The body (100) and the cap (104) are combined to form a capsule. The composition of the capsules is typically a gelatinous substance which is hardened into a shell which is then usable for holding a dry, powdered ingredients or miniature pellets and any other combinations thereof. Capsules can also have a soft shell, which can be but is not limited to oils for active ingredients that are dissolved or suspended or a harder shell that remains dissolvable when consumed. The type of substances utilized for the creation of such capsules is well known in the art and needs no further elaboration herein.

### SUMMARY OF THE INVENTION

The invention relates generally to the field of a two-piece capsules and is more particularly, but not by way of limitation, directed to methods and apparatus for an air-expelling and bubble-free capsule. In particular, the invention disclosed herein is a method and apparatus for a capsule with a consumable insert that is configured to expel air bubbles from the composition inside the capsule. In addition, the rounded flattened shape of the cap section of the capsule allows for further removing or substantially reducing of the air bubble or meniscus in liquid-filled capsules.

A two-piece capsule cap for powder-filled capsules, comprising, a) a cap, wherein the cap has a flattened shape with rounded edges, b) a body, wherein the body is configured to fit inside the cap and d) at least one adjoining element, wherein the at least one adjoining element is located on underside of the cap, wherein the at least one adjoining element is configured to allow a top edge of the body to enter the at least one adjoining element, wherein the body is configured to sit inside the at least one adjoining element thereby establishing a fit between the body and the cap. It should be noted that the adjoining element b) is a slit within the cap. In addition, the adjoining element b) is a notch within the cap. The body b) mentioned above is elongated and in addition the cap a) has a wider diameter than the body thereby creating an overlap between the body and the cap. Additionally, body b) is filled with a powder and the cap is attached, enabling the body to hold a volume of the powder and minimize air within the body.

In another embodiment, disclosed herein is a two-piece air-expelling and bubble-free capsule comprising, a) a cap coupled to a body portion of a capsule b), where the body containing a composition for consumption where the body is configured to fit inside the cap. In addition, c) an insert positioned within the cap, where the insert further comprises a a) a curved side configured to compress against a meniscus of the composition for consumption and thereby reduce air from the composition, b) a flat side which sits underside of the cap. It should be noted that the cap a) is a solid and the body b) body is elongated. In addition, the insert c) is a solid composition able to compress against the composition for consumption, where the insert is having a dome shape. The cap a) has a wider diameter than the body, wherein the body fits inside the cap thereby creating an overlap between the body and the cap. Additionally, d) a top edge of the body contacts with the cap, where the top edge of the body sits inside a junction between the insert and the cap.

In another embodiment, disclosed herein is a two-piece air-expelling and bubble-free capsule comprising, a) a cap, b) a body, where the body is configured to fit inside the cap. In addition, c) an insert positioned within the cap, the insert having a dome shape, the insert further comprising, a) a curved side configured to compress against the composition for consumption and thereby expelling air from the composition. Also, the insert includes b) a flat side of the insert, where the flat side of insert is attached to an underside of cap, and d) a sealing element, wherein the cap and the body are sealed via a sealing element, wherein the sealing element prevents leakage of the composition. It should be noted that the body b) is elongated. The cap a) has a wider diameter than the body, wherein the body fits inside the cap thereby creating an overlap between the body and the cap. Additionally, c) a top edge of the body contacts the cap, wherein the top edge of body sits inside a junction between the insert and the cap.

In another embodiment, disclosed herein is a method for the encapsulation of a two-piece, liquid-filled, bubble-free capsule comprising, a) separating a cap from a body, b) rotating the cap approximately 180 degrees, opening of the cap faces up, c) placing the cap into a liquid-filling machine, d) dosing a formulated plug of a material being encapsulated into the cap, where the formulated plug will solidify forming a dome, and the dome faces toward the opening of the cap. Furthermore, d) rotating the cap approximately 180 degrees, and the cap faces right-side up. Additionally, e) applying a sealing solution to a lower inner surface of the cap and f) filling the body with a viscous composition. Additionally, g) mating the cap to the body, where a filled capsule is formed and i) discharging the filled capsule from the liquid-filling machine. It should be noted that approximately 100% utilization of internal volume in the capsule is achieved. In addition, the utilization of the internal volume of the capsule results in at least approximately 15 % increase in a fill weight. Additionally, the viscous composition dissolves the formulated plug deposited into the cap.

### BRIEF DESCRIPTION OF DRAWINGS

FIG. 1 illustrates a prior art capsule.

FIG. 2 illustrates a two-piece capsule cap for powder-filled capsules configured in accordance with one or more embodiments of the invention.

FIG. 3 illustrates a illustrates a two-piece air-expelling and bubble-free capsule configured in accordance with one or more embodiments of the invention.

FIG. 4 illustrates a two-piece air-expelling and bubble-free capsule with a sealing element configured in accordance with one or more embodiments of the invention.

FIG. 5 illustrates a method for the encapsulation of a two-piece, liquid-filled, bubble-free capsule configured in accordance with one or more embodiments of the invention.

### DETAILED DESCRIPTION

A method and apparatus for a two-piece capsule configured to expel air from the interior portion of the capsule will now be described in accordance with one or more embodiments of the invention. In the following exemplary description numerous specific details are set forth to provide a more thorough understanding of embodiments of the invention. It will be apparent, however, to an artisan of ordinary skill that the present invention may be practiced without incorporating all aspects of the specific details described herein. Furthermore, although steps or processes are set forth in an exemplary order to provide an understanding of one or more systems and methods, the exemplary order is not meant to be limiting. One of ordinary skill in the art would recognize that the steps or processes may be performed in a different order, and that one or more steps or processes may be performed simultaneously or in multiple process flows without departing from the spirit or the scope of the invention. In other instances, specific features, quantities, or measurements well known to those of ordinary skill in the art have not been described in detail so as not to obscure the invention. It should be noted that although examples of the invention are set forth herein, the claims, and the full scope of any equivalents, are what define the metes and bounds of the invention.

For a better understanding of the disclosed embodiment, its operating advantages, and the specified object attained by its uses, reference should be made to the accompanying drawings and descriptive matter in which there are illustrated exemplary disclosed embodiments. The disclosed embodiments are not intended to be limited to the specific forms set forth herein. It is understood that various omissions and substitutions of equivalents are contemplated as circumstances may suggest or render expedient, but these are intended to cover the application or implementation.

The term “first”, “second” and the like, herein do not denote any order, quantity, or importance, but rather are used to distinguish one element from another, and the terms “a” and “an” herein do not denote a limitation of quantity, but rather denote the presence of at least one of the referenced items.

Spatially relative terms, such as “beneath,” “below,” “lower,” “under,” “above,” “upper,” and the like, may be used herein for ease of explanation to describe one element or feature's relationship to another element(s) or feature(s) as illustrated in the figures. It will be understood that the spatially relative terms are intended to encompass different orientations of the device in use or in operation, in addition to the orientation depicted in the figures. For example, if the device in the figures is turned over, elements described as “below” or “beneath” or “under” other elements or features would then be oriented “above” the other elements or features. Thus, the example terms “below” and “under” can encompass both an orientation of above and below. The device may be otherwise oriented (e.g., rotated 90 degrees or at other orientations) and the spatially relative descriptors used herein should be interpreted accordingly.

It will be understood that when an element or layer is referred to as being “on,” “connected to,” or “coupled to” another element or layer, it can be directly on, connected to, or coupled to the other element or layer, or one or more intervening elements or layers may be present. In addition, it will also be understood that when an element or layer is referred to as being “between” two elements or layers, it can be the only element or layer between the two elements or layers, or one or more intervening elements or layers may also be present.

As used herein, the term “substantially,” “about,” and similar terms are used as terms of approximation and not as terms of degree, and are intended to account for the inherent deviations in measured or calculated values that would be recognized by those of ordinary skill in the art. Further, the use of “may” when describing embodiments of the present invention refers to “one or more embodiments of the present invention.” As used herein, the terms “use,” “using,” and “used” may be considered synonymous with the terms “utilize,” “utilizing,” and “utilized,” respectively. Also, the term “exemplary” is intended to refer to an example or illustration.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

References. The description of the figures provided herein contains references to each depicted component. A list of these components described in the context of each figure is provided below for easy reference.
100 Body
102 Overlap
104 Cap
106 Top Edge of the Body
108 Underside of the Cap
200 Adjoining Element
202 Rounded Edges of the Cap
204 Flattened Cap
300 Insert
304 Flat Side of the Insert
308 Curved Side of the Insert
400 Sealing Element
500 Separating the Cap from The Body
502 Rotating Cap 180 Degrees
504 Placing the Cap in a Liquid-Filling Machine
506 Dosing a Formulated Plug
508 Rotating Cap 180
510 Applying a Sealing Solution to the Lower Inner Surface of the Cap
512 Filing body with Viscous Composition
514 Mating Cap to the Body
516 Discharging the Capsule from the Liquid-Filling Machine

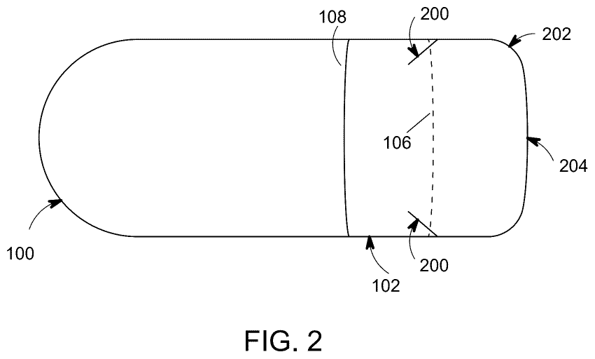

FIG. 2 illustrates the components of a two-piece capsule utilized for powders or an equivalent composition, powder-filled capsules as configured in accordance with one or more embodiments of the invention. The two-piece capsule for powder-filled capsules is made up of a body (100), the top edge of the body (100), the underside of the cap (108), a flattened cap (204) with rounded edges of the cap (202), at least one adjoining element (200), and an overlap (102) where the body (100) fits inside the flattened cap (204). Similarly, and still in keeping with the scope and spirit of the invention, the body may optionally have a wider diameter than the cap so that the cap fits within the body. In either configuration the two components are combined to form a capsule. At least one adjoining element (200), is located on the underside of the flattened cap (204), and is configured to allow the top edge of body (106) to enter inside at least one adjoining element (200), which can be but is not limited to a locking element or notch, or a slit, or an opening.

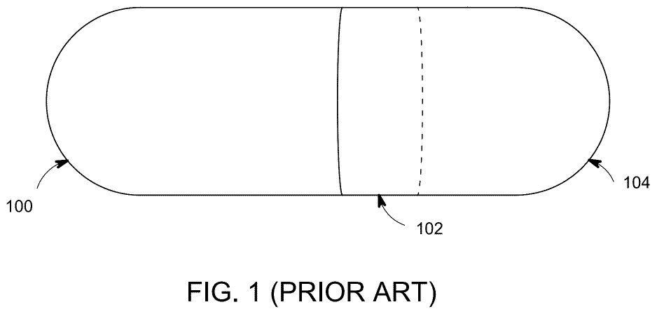

In addition, the body (100) in FIG. 2 is configured to sit inside the at least one adjoining element (200) thereby establishing a fit between the body (100) and the flattened cap (204). The flattened cap (204) is also configured to eliminate the type of denting that sometimes occurs when the cap (104) is round similar to the cap (104) shown in FIG. 1. In addition, in the prior art shown in FIG. 1, when the prior art capsule is filled with powder, a gap is created since the powder is not filled completely to the top of the cap (104) thereby creating an airspace inside the cap (104). This airspace results in excess air (oxygen and water vapor). Furthermore, in addition to the airspace in the cap (104), air is also trapped inside the capsule when the body (100) and the cap (104) are combined to form a capsule. The air inside the capsule gets distributed within the powder. The trapped air inside the capsule affects the stability of the contents via degradation of the active ingredients'contents inside the capsule because every single particle of the powder is in contact with the air (oxygen and water vapor). Using a flattened cap (204), which is called for in one or more embodiments of the invention, instead of a round cap (104), reduces trapped air from being held within the capsule. In addition, the flattened cap (204) reduces the risk of indentation (dented cap). The edges of the flattened cap (204) are typically rounded in order to allow the user of the capsule to more easily swallow the capsule without encountering sharp edges. It should be noted that the flattened cap (204) embodiment shown in FIG. 2 does not result in a lower volume of powder when compared to the embodiment in FIG. 1. This is because the capsule in FIG. 1 is not filled to the top of the body (104) thereby creating an airspace. By employing a flattened cap (204), the gap or airspace is reduced from the cap (104) and the dead space or gap is reduced and as a result, the overall volume of the capsule is unchanged. Resulting reduction of oxygen and water vapor retards degradation of the active ingredients, thereby prolonging the shelf life of the product.

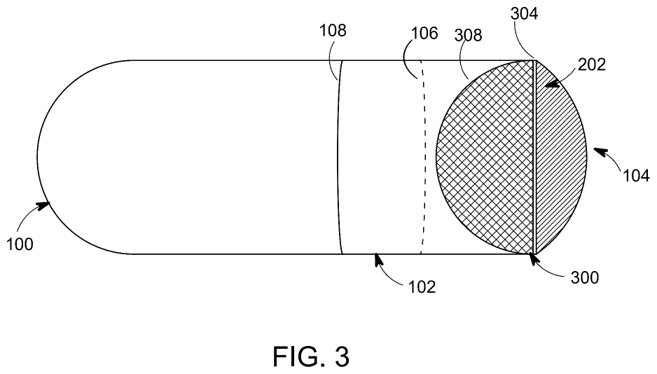

FIG. 3 illustrates the components of a two-piece air-expelling and bubble-free capsule as configured in accordance with one or more embodiments of the invention. The two-piece capsule is made up of a body (100), the top edge of the body (106), the underside of the cap (108) which can be but is not limited to a rigid or a semi-rigid consumable solid composition or shape, a cap (104) which also can be but is not limited to a rigid or a semi-rigid consumable solid composition or shape, an overlap (102) where the body (100) fits inside the cap (104) or visa-versa, and an insert (300) which has a curved side and a flat side. In addition, the insert (300) can be but is not limited to a solid or a rigid dome or an equivalent shape, where it can be consumed by a person. It should be noted that the flat side of the insert (304) interfaces with the underside of the cap (104). The cap (104) in this figure can be smaller compared to the cap (104) shown in FIG. 1 in order to minimize trapped air inside the cap (104). It should be noted that the top edge of the body (100) contacts with the cap (104) and sits inside a junction between end of the insert (300) and the cap (104).

In an embodiment of the invention where the capsule is filled with a liquid, the liquid is not filled to the top of the body, thereby creating a meniscus, which contains air (oxygen and water vapor). The meniscus inside the capsule can cause overall degradation and affect the stability of the liquid inside the capsule. Traditionally gas such as Nitrogen was used to purge trapped air from the composition within the capsule. To reduce the meniscus, a cap (104), which can be shaped as a concave or convex dome, can be used to reduce the meniscus inside the capsule. The cap (104) shown here can be physically smaller in size compared to the cap shown in FIG. 1. Although the cap (104) reduces the meniscus inside the capsule, it does not completely eliminate all air within the capsule because of the small curvature that still exists in the cap (104). The small or reduced curvature in the cap (104) is helpful to allow consumers to more a user can easily swallow the capsule. To further reduce the remaining bubble from the liquid-filled capsule, an additional insert (300), which can be but is not limited to a solid or a rigid dome or an equivalent shape, may be used. The curved side of the insert (308) is configured to mate with the liquid meniscus thereby minimizing bubble formation inside the body (100). The flat side of the insert (304) is attached to the underside of the cap (108) such that the insert (300) fills the remaining empty space created by the curvature of the cap (108). By attaching the flat side of the insert (304) to the underside of the cap (108), the insert (300) will mate with or compress against the meniscus thereby reducing it from the capsule. By employing the use of the insert (304) and the cap (108), the trapped air is reduced from the capsule and gas such as Nitrogen used to purge air from the bubble is not required.

In another embodiment of the invention the capsule is filled with a powder or an equivalent material. In this embodiment the insert (300) and the cap (108) work together to expel air trapped inside the powder in the capsule body (100). As discussed, air (oxygen and water vapor) trapped inside the capsule gets distributed within the powder which leads to degradation of the powder inside the capsule body (100) because every single particle of the powder or equivalent material is in contact with the trapped air (oxygen and water vapor). To expel a fraction of air contained in the powder (oxygen and water vapor) in the capsule body (100), the insert (300) is used to compress the powder inside the capsule, causing air (oxygen and water vapor) to be substantially expelled from the capsule thereby resulting in a product with potentially stability and greater shelf life.

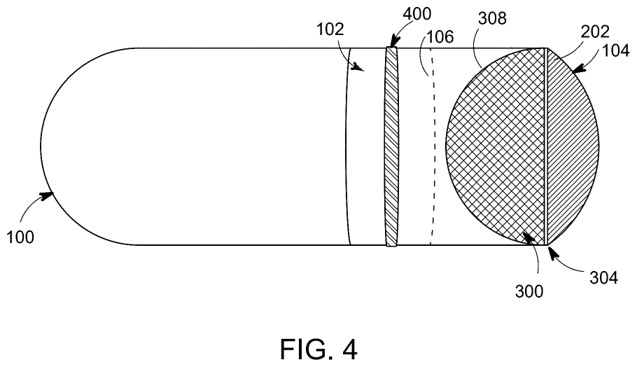

FIG. 4 illustrates a two-piece bubble-free capsule with a sealing element in accordance with one or more embodiments of the invention. The two-piece bubble-free capsule is made up of a body (100), a cap (104) which can be but is not limited to a rigid or a semi-rigid consumable solid composition or shape, an overlap (102) where the body (100) fits inside the cap (104), and an insert (300), which has a curved side of the insert (308) and a flat side of the insert (304). In addition, the insert (300) can be but is not limited to a dome, where it can be consumed and can be a rigid or a semi-rigid solid composition or shape. It should be noted that the flat side of the insert (304) interfaces with the underside of the cap (108). Additionally, the cap (104) shown here can be physically smaller in size, and arch compared to the cap (104) shown in FIG. 1. In addition, the body (100) has a top edge of the body (106) contacts with the cap (104) and sits inside a junction between the end of the insert (300) and the cap (104).

The capsule shown in FIG. 4 can be filled with either powder or liquid. When the capsule is filled with liquid, a sealing element (400) is used in order to seal the capsule and not allow the liquid contents to spill out of the capsule. It should be noted that the sealing element (400) can also be used when powder is placed inside the capsule body (100) because in this instance the sealing element (400) prevents the powder from leaking out of the capsule and provides a measure of tamper-evidence.

Traditionally, removing trapped air within a capsule is achieved by a process using a Nitrogen-Purge Instant Bonding (NPIB) System where a cap (104) and a body (100) are combined via a melted surface where the cap (104) melts with the surface of the body (100). This NPIB process is not required when embodiments of the invention such as those set forth herein are utilized.

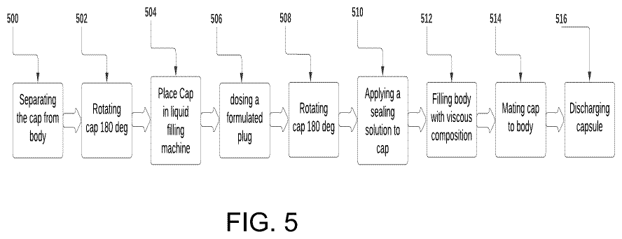

FIG. 5 illustrates a method for the encapsulation of a two-piece, liquid-filled, bubble-free capsule in accordance with one or more embodiments of the invention. As discussed in FIGS. 3 and 4, a two-piece, liquid-filled, bubble-free capsule using a cap (104) is mated to an insert (300) as described in FIGS. 3 and 4 such that the meniscus is minimized from the capsule body (100). As a result, there is no need for a gas such as Nitrogen or any other gas to purge air from the capsule.

To achieve the two-piece, liquid-filled, bubble-free capsule the cap is first separated (500) from the body (100). The cap (104) is then rotated (502) approximately 180 degrees so the cap (104) opening faces up. The next step requires placing (504) the upside-down cap (104) in a liquid-filling machine or an equivalent device. The next step involves dosing (506) a formulated plug of the material being encapsulated into the domed section of the cap (104). It should be noted that the formulation will solidify almost instantly, forming a dome or equivalent shape facing toward the open end of the cap (104). The cap is then rotated (508) again at approximately 180 degrees or about 180 degrees where the cap (104) is now facing right-side up. A sealing solution is applied (510) to the lower inner surface of the cap (104). After, the cap (104) is mated (514) to the body (100), which was pre-filled with a viscous composition (512) that can be either a mobile or a semi-viscous version of the composition present in the cap (104). After the cap (104) is mated (514) to the body (100), the liquid-filled body and cap are sealed and discharged (516) from the capsule-filling machine, thereby resulting in a bubble-free liquid-filled capsule.

It should be noted that the two-piece, liquid-filled, bubble-free capsule described in FIGS. 3, 4 and 5 achieves better utilization of the internal volume of the capsule than prior art approaches and will also result in an increase in the amount of liquid composition filled into the capsule.

Thus, a method and apparatus of a two-piece capsule, air-expelling and bubble-free capsule is described. The reader should note however that the claims set forth below and the full scope of their equivalents are what define the scope of the invention.

1. A two-piece capsule cap for powder-filled capsules, comprising:
a cap having a wider diameter than an elongated body, said cap has having a flattened shape with rounded edges; and
said elongated body having a greater volume than said cap to contain ingredients for consumption, wherein said elongated body is configured to fit inside said cap to create a sealed unit.

2. (canceled)

3. (canceled)

4. (canceled)

5. (canceled)

6. The body of claim 1, wherein said body is filled with a powder and said cap is attached enabling said body to hold a volume of said powder and minimize air within said body.

7. A two-piece bubble-free capsule comprising;
a solid cap coupled to a body of a capsule, said cap having no air pocket;
said body containing a liquid composition for consumption, where said body is configured to fit inside said cap;
an insert positioned within said cap, wherein said insert is a semi-rigid consumable solid which displaces air by covering a meniscus of the liquid composition;
said insert further comprising:
a curved side configured to compress against said meniscus of said liquid composition for consumption and thereby reducing air from said liquid composition; and
a flat side which sits underside of said cap.

8. (canceled)

9. The body of claim 7, wherein said body is elongated.

10. The insert of claim 7, wherein said insert is configured to compress against said liquid composition for consumption, wherein said insert has a dome shape.

11. The cap of claim 7, wherein said cap has a wider diameter than said body, wherein said body fits inside said cap thereby creating an overlap between said body and said cap.

12. The body of claim 7, wherein a top edge of said body contacts with said cap, wherein said top edge of said body sits inside a junction between said insert and said cap.

13. A two-piece air expelling capsule comprising;
a cap, wherein said cap is a solid;
a body, wherein said body is configured to fit inside said cap;
an insert positioned within said cap, said insert having a dome shape, wherein said insert is a semi-rigid consumable solid, said insert further comprising:
a curved side configured to compress against a composition for consumption and thereby expelling air from said composition;
a flat side of said insert, wherein said flat side of said insert is attached to an underside of said cap; and
a sealing element, wherein said cap and said body are sealed via a sealing element, wherein said sealing element prevents leakage of said composition.

14. The body of claim 13, wherein said body is elongated.

15. The cap of claim 13, wherein said cap has a wider diameter than said body, wherein said body fits inside said cap thereby creating an overlap between said body and said cap.

16. The body of claim 13, wherein a top edge of said body contacts said cap, wherein said top edge of body sits inside a junction between said insert and said cap.

17. A method for the encapsulation of a two-piece, liquid filled, bubble-free capsule comprising:
separating a cap from a body;
rotating said cap approximately 180 degrees, wherein opening of said cap faces up;
placing said cap into a liquid-filling machine;
placing a formulated plug into said cap,
rotating said cap approximately 180 degrees, wherein said cap faces right-side up;
filling said body with a a viscous composition;
applying a sealing solution to an lower inner surface of said cap;
mating said formulated plug of said cap with said viscous composition such that said formulated plug comes in contact with a meniscus of said viscous composition inside said body;
sealing said filled capsule; and
discharging said filled capsule from said liquid-filling machine.

18. The filled capsule of claim 17, wherein up to approximately 100% utilization of internal volume in said capsule is achieved.

19. The utilization of internal volume of claim 18, wherein said up to approximately 100 % utilization of said internal volume of said capsule results in at least approximately 15 % increase in a fill weight.

20. (canceled)

21. A two-piece air expelling capsule comprising;
a cap, wherein said cap is a solid;
a body, wherein said body is configured to fit inside said cap;
an insert positioned within said cap, said insert having a dome shape, wherein said insert is a rigid consumable solid, said insert further comprising:
a curved side configured to compress against a composition for consumption and thereby expelling air from said composition;
a flat side of said insert, wherein said flat side of said insert is attached to an underside of said cap; and
a sealing element, wherein said cap and said body are sealed via a sealing element. wherein said sealing element prevents leakage of said composition.
